(19) United States
(12) Patent Application Publication
(10) Pub. No.: US 2024/0374245 A1
(43) Pub. Date: Nov. 14, 2024

(54) METHOD AND APPARATUS FOR BREAST BIOPSY

(71) Applicant: KOREA INSTITUTE OF SCIENCE AND TECHNOLOGY, Seoul (KR)

(72) Inventors: Sunghwan LIM, Seoul (KR); Sangseo JEON, Seoul (KR); Hyehyun HAN, Seoul (KR)

(73) Assignee: KOREA INSTITUTE OF SCIENCE AND TECHNOLOGY, Seoul (KR)

(21) Appl. No.: 18/649,418

(22) Filed: Apr. 29, 2024

(30) Foreign Application Priority Data
May 11, 2023 (KR) 10-2023-0061340

(51) Int. Cl. A61B 10/00; A61B 5/107; A61B 34/10; A61B 34/20; G06T 3/40; G06T 7/00; G06T 7/11; G06T 7/20; G06T 7/30; G06T 7/50; G06T 17/00

(52) U.S. Cl. CPC A61B 10/0041; A61B 5/1075; A61B 34/10; A61B 34/20; G06T 3/40; G06T 7/0012; G06T 7/11; G06T 7/20; G06T 7/30; G06T 7/50; G06T 17/00; A61B 2034/105; G06T 2200/04; G06T 2207/10088; G06T 2207/30068; G06T 2207/30096; G06T 2210/41

(57) ABSTRACT

A method for a breast biopsy based on indirect magnetic resonance imaging (MRI) guidance of present disclosure, may comprises obtaining a breast MRI data of a patient, generating a deformable breast model using the breast MRI data, measuring real-time breast shape of the patient using a depth sensor, performing a real-time deformable registration using the deformable breast model and the real-time shape data, estimating movements of the MRI targets inside of the breast, performing a breast biopsy for the MRI targets.

### CROSS-REFERENCE TO RELATED APPLICATION

This application claims priority to and the benefit of Korean Patent Application No. 10-2023-0061340 filed in the Korean Intellectual Property Office on May 11, 2023 the entire contents of which are incorporated herein by reference.

### BACKGROUND OF THE INVENTION

### Field of the Invention

The present disclosure relates to a method and apparatus for a breast biopsy, and in particular to a method and apparatus for a breast biopsy based on indirect magnetic resonance imaging (MRI) guidance.

### Description of the Related Art

Breast cancer is the most prevalent cancer in the world and is the cancer that accounts for the highest proportion of cancer deaths among women worldwide. According to the World Health Organization (WHO), it is reported that approximately 2.3 million women have been diagnosed with breast cancer, and approximately 700,000 people have died from breast cancer in 2020 alone.

Symptoms of breast cancer generally include changes in the shape or size of the breast, a lump within the breast, etc. Patients with these symptoms generally undergo basic tests such as physical examination, mammography, ultrasound, or breast MRI, but a breast biopsy using a physical needle is essential for a final diagnosis of breast cancer.

Such breast biopsies are generally performed using mammograms, ultrasound images, MRI, etc. Among these, breast MRI is known to have superior sensitivity and specificity for identifying lesions suspected of being cancer compared to other medical images.

As a method of performing breast biopsy using MRI data, direct MRI-guided breast biopsy is used, in which the biopsy is performed within an MRI scanner located in an MRI room. However, direct MRI-guided breast biopsy has limitations such as high cost, long lead time, narrow space, and the inability to accompany conventional X-ray or ultrasound image-guided breast biopsy.

To solve these problems, research is required on indirect MRI-guided breast biopsy, which can perform breast biopsy based on MRI data outside the MRI room.

### SUMMARY OF THE INVENTION

The present disclosure may provide a method and apparatus for performing a breast biopsy based on indirect MRI guidance.

The present disclosure provides a method and apparatus for performing a breast biopsy based on indirect MRI guidance, which can solve problems with a breast biopsy based on direct MRI guidance.

The present disclosure provides a method and apparatus for performing a breast biopsy based on indirect MRI guidance, which can increase the detection rate of breast cancer.

A method for a breast biopsy based on indirect magnetic resonance imaging (MRI) guidance of present disclosure, may comprises obtaining a breast MRI data of a patient, generating a deformable breast model using the breast MRI data, measuring real-time breast shape of the patient using a depth sensor, performing a real-time deformable registration using the deformable breast model and the real-time shape data, estimating movements of the MRI targets inside of the breast, performing a breast biopsy for the MRI targets.

The generating of the deformable breast model may include segmenting breast tissue of the patient from the breast MRI data and representing the breast of the patient as a three-dimensional (3D) model by allocating a plurality of volume elements to the segmented breast tissue.

Different numbers of volume elements may be allocated to each area of the breast tissue based on accuracy required for an examination.

Each of the plurality of volume elements may be given a governing equation based on elastic potential energy, and the governing equation may include material parameters of the breast tissue.

The method may further comprise, updating material parameters of the deformable breast model in real time based on interaction between a medical instrument and a breast of the patient, wherein the update may be performed based on a contact area between the medical instrument and the breast.

The material parameters of the deformable breast model may be updated based on a difference between first surface information measured in real time in an area other than the contact area and second surface information calculated from an area other than a contact area on the deformable breast model.

The difference between the first surface information and the second surface information may be calculated based on Hausdorff distance.

The real-time deformable breast model may be updated based on information collected about a breast of the patient from various angles by the depth sensor.

The information collected about the breast of the patient from various angles may include information about blind spots where visibility is obscured.

The information collected about the breast of the patient from various angles may be obtained by changing a position and orientation of the depth sensor in real time.

The method may further comprise performing deformable registration between the deformable breast model generated from the MRI data and the real-time breast shape data measured by the depth sensor.

The registration between the two data may be performed separately into rigid-body registration and real-time deformation registration.

The measuring real-time breast shape may be performed by the depth sensor acquiring breast surface information of the patient and generating and updating a deformable surface fusion model in real time.

The deformable surface fusion model may be obtained by calculating a degree of deformation between a previous unit time and a current unit time for each area.

The deformable surface fusion model may be obtained by giving higher spatial resolution to areas with a large degree of deformation than to areas with a relatively small degree of deformation.

An apparatus for an indirect MRI-guided breast biopsy of the present disclosure may comprise a transceiver configured to transmit and receive a signal, a processor configured to control the transceiver.

The processor may be configured to, obtain a breast MRI data of a patient, generating a deformable breast model using the breast MRI data, measure real-time breast shape of the patient using a depth sensor, perform a real-time deformable registration using the deformable breast model and the real-time shape data, estimating movements of the MRI targets inside of the breast, perform a breast biopsy for the MRI targets.

The deformable breast model may be generated by segmenting breast tissue of the patient from the breast MRI data, and representing the breast of the patient as a 3D model by allocating a plurality of volume elements to the segmented breast tissue.

Different numbers of volume elements may be allocated to each area of the breast tissue based on accuracy required for an examination.

The processor may update material parameters of the deformable breast model in real time based on interaction between a medical instrument and a breast of the patient, wherein the update may be performed based on a contact area between the medical instrument and the breast.

The real-time deformable breast model is updated based on information collected about a breast of the patient from various angles by the depth sensor.

### BRIEF DESCRIPTION OF THE DRAWINGS

FIG. 1 is a diagram schematically illustrating a method for an indirect MRI-guided breast biopsy according to an embodiment of the present disclosure.

FIG. 2 is a diagram for explaining a method of generating a deformable breast model according to another embodiment of the present disclosure.

FIG. 3 is another diagram for explaining a method for generating a real-time deformable breast model.

FIG. 4 is a diagram for explaining a method of updating deformable breast model parameters according to another embodiment of the present disclosure.

FIG. 5 is another diagram for explaining a method of updating deformable breast model parameters.

FIG. 6 is a diagram for explaining a method of measuring breast deformation using a depth sensor according to another embodiment of the present disclosure.

FIG. 7 is a diagram for explaining an image-to-patient registration method according to another embodiment of the present disclosure.

FIG. 8 a diagram for explaining an apparatus for an indirect MRI-guided breast biopsy according to another embodiment of the present disclosure.

FIG. 9 is a diagram for explaining an apparatus for an indirect MRI-guided breast biopsy according to another embodiment of the present disclosure.

FIG. 10 is a diagram for explaining an apparatus for an indirect MRI-guided breast biopsy according to another embodiment of the present disclosure.

FIG. 11 is a diagram for explaining an apparatus for an indirect MRI-guided breast biopsy according to another embodiment of the present disclosure.

FIG. 12 is a diagram illustrating a configuration of an apparatus according to an embodiment of the present disclosure.

### DETAILED DESCRIPTION OF THE EMBODIMENTS

Hereinafter, with reference to the attached drawings, embodiments of the present disclosure will be described in detail so that those skilled in the art can easily practice them. However, the present disclosure may be implemented in many different forms and is not limited to the embodiments described herein.

In describing embodiments of the present disclosure, if it is determined that a detailed description of a known configuration or function may obscure the gist of the present disclosure, the detailed description thereof will be omitted. In addition, in the drawings, parts that are not related to the description of the present disclosure are omitted, and similar parts are given similar reference numerals.

In the present disclosure, when a component is said to be “connected,” “coupled,” or “be in contact with” to another component, this may include not only to direct connections, but also to indirect connections where another component exists in between. In addition, when a component is said to “include” or “have” another component, this does not mean that other components are excluded, but that other components can be further included, unless specifically stated to the contrary.

In the present disclosure, terms such as first, second, etc. are used only for the purpose of distinguishing one component from other components, and do not limit the order or importance between components unless specifically mentioned. Therefore, within the scope of the present disclosure, a first component in one embodiment may be referred to as a second component in another embodiment, and similarly, the second component in one embodiment may be referred to as a first component in another embodiment.

In the present disclosure, distinct components are intended to clearly explain each feature, and do not necessarily mean that the components are separated. That is, a plurality of components may be integrated to form one hardware or software unit, or one component may be distributed to form a plurality of hardware or software units. Accordingly, even if not specifically mentioned, such integrated or distributed embodiments are also included in the scope of the present disclosure.

In the present disclosure, components described in various embodiments do not necessarily mean essential components, and some may be optional components. Accordingly, embodiments consisting of a subset of the elements described in one embodiment are also included in the scope of the present disclosure. Additionally, embodiments that include other components in addition to the components described in the various embodiments are also included in the scope of the present disclosure.

The advantages and features of the present disclosure, and methods for achieving them will become clear by referring to the embodiments described in detail below along with the accompanying drawings. However, the present disclosure is not limited to the embodiments presented below and may be implemented in various forms, and these embodiments are provided solely to ensure that the present disclosure is complete and to fully inform those skilled in the art of the invention of the scope of the invention.

The terms used in the present disclosure are only used to describe specific embodiments and are not intended to limit the present disclosure. Singular expressions include plural expressions unless the context clearly indicates otherwise. In the present disclosure, terms such as “comprise” or “include” are intended to designate the presence of described features, numbers, steps, operations, components, parts, or combinations thereof, and it should be understood that this does not exclude in advance the presence or addition of one or more other features, numbers, steps, operations, components, parts, or combinations thereof.

Unless specifically defined otherwise, all terms used herein, including technical or scientific terms, have the same meaning as generally understood by a person of ordinary skill in the technical field to which the present disclosure pertains. Terms such as those defined in commonly used dictionaries should be interpreted as having a meaning consistent with the meaning in the context of the related technology, and unless clearly defined in the present disclosure, should not be interpreted in an idealized or excessively formal meaning.

Hereinafter, a method and apparatus for an indirect MRI-guided breast biopsy according to the present disclosure will be described in detail with reference to the drawings.

Symptoms of breast cancer may generally appear in the form of changes in the shape or size of the breast or a lump in the breast. Patients with breast cancer symptoms undergo basic examinations such as physical examination, mammography, X-ray examination, and breast MRI, but a biopsy using a physical needle is essential for the final diagnosis of breast cancer.

Because of the fatality of breast cancer, there is a great need for early detection of breast cancer through early diagnosis. If breast cancer is small and confined to the breast, patients can choose from a variety of treatment methods to increase survival rates. The generally preferred method for diagnosing breast cancer is core needle breast biopsy, which involves taking tissue samples from lesions suspected of being breast cancer using a specimen collection needle. Such breast biopsy can be performed using mammograms, ultrasound images, and MRI.

In particular, breast biopsy using X-ray and ultrasound images is widely used as a general method for diagnosing breast cancer. However, these methods have the problem that they cannot visualize images in real time and can only visualize specific substances and relatively large lesions. In contrast, breast MRI are reported to have superior sensitivity and specificity for identifying lesions suspected of being cancer compared to other medical images. Breast MRI is known to be particularly good at identifying aggressive and invasive early tumors. In fact, it is known that more than half of breast cancer can be detected only through breast MRI.

Conventionally, in order to effectively utilize the above-mentioned advantages of breast MRI, direct MRI-guided breast biopsy, in which breast biopsy is performed directly in an MRI room, has been used. Direct MRI-guided breast biopsy is performed in an MRI scanner located in an MRI room and can be performed by taking an MRI of the patient's breast fixed in the apparatus, identifying lesions, and then immediately collecting a tissue sample using a core needle.

Direct MRI-guided breast biopsy has the advantage of being able to directly visualize the area suspected of breast cancer, but it has limitations as it can only be performed under limited resources and space. Specifically, direct MRI-guided biopsy has problems such as high cost, long lead time, difficulty in space utilization, and the inability to simultaneously perform X-ray and ultrasound image-guided biopsy.

According to the indirect MRI-guided biopsy proposed by the present disclosure, breast biopsy based on MRI data is possible outside the MRI room, so the above-mentioned problems can be solved, and the detection rate of breast cancer can be greatly increased.

### Indirect MRI-Guided Breast Biopsy Method

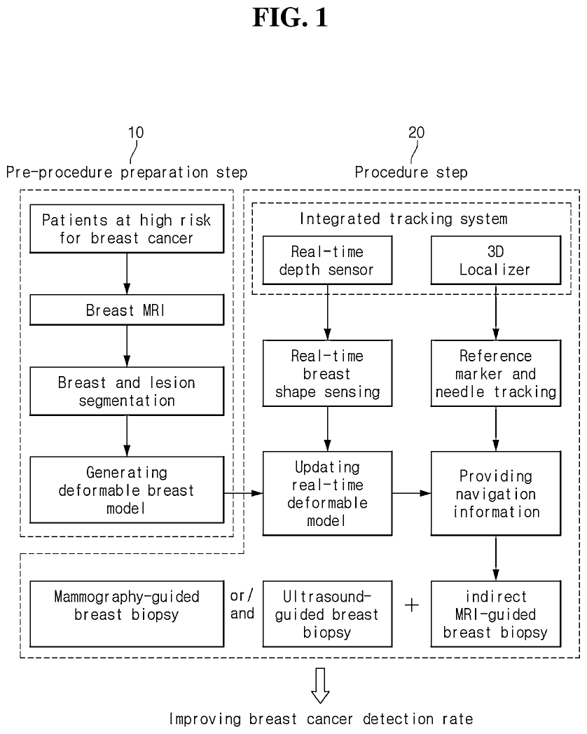

FIG. 1 is a diagram schematically illustrating a method for an indirect MRI-guided breast biopsy according to an embodiment of the present disclosure.

According to an embodiment of the present disclosure, indirect MRI-guided biopsy may be divided into a pre-procedure preparation step 10 and a surgical procedure step 20. The pre-procedure preparation step 10 may be a step of taking breast MRI of patients at high risk for breast cancer. Breast MRI data can be used to determine whether a patient has lesions suspicious for breast cancer.

A breast MRI data of a patient with a lesion suspected to be breast cancer can be used to segment the breast and lesion areas. A 3D breast model can be generated using the segmented MRI data. Here, the 3D breast model may include at least one of a breast model and a lesion model. Afterwards, a deformable breast model can be generated using the 3D models.

Indirect MRI-guided breast biopsy in the surgical procedure step 20 may be performed using a depth sensor and a 3D localizer. In the surgical procedure step 20, the changes in the external appearance of the patient's breast and the 3D positions of the medical instrument and its guide apparatus can be tracked in real time using integrated tracking apparatus.

Real-time deformable registration can be performed using real-time shape sensing data of the patient's breast obtained using a depth sensor and a deformable breast model generated in the pre-procedure stage. Through real-time deformable registration, the position of at least one of the breast, lesion area, medical instrument, and medical instrument guide apparatus can be calculated in real time. Position information calculated in real time can be provided to apparatus such as external monitors, Head Mounted Display (HMD) apparatus, surgical robots, robotic arms, and biopsy apparatus.

Based on information provided in real time, the operator, or the indirect MRI-guided biopsy apparatus can perform a biopsy on the lesion area or target area of the breast.

The above-described indirect MRI-guided biopsy method can be performed outside the MRI room. Additionally, the indirect MRI-guided biopsy method can be performed in parallel with conventional X-ray and ultrasound image-guided biopsy, which can increase the early detection rate of breast cancer.

### Method for Generating Deformable Breast Model

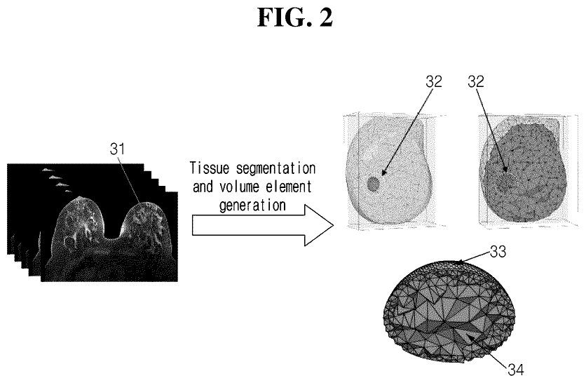

FIG. 2 is a diagram explaining a method of generating a deformable breast model according to another embodiment of the present disclosure.

Hereinafter, the method for generating a deformable breast model presented in FIG. 1 will be described in detail. In some embodiments of the present disclosure, the deformable breast model can be used to presume in real time the change in position of the tissue presumed to be the internal lesion 31 according to the external shape of the breast.

A method of segmenting breast region from a patient's breast MRI data to generate a 3D deformable breast model may be presented. Here, different material property information may be given to the tissue 32 presumed to be a lesion or a tissue that has different material properties from the surrounding tissue. The breast region can be segmented into distinct tissue regions, allowing for the application of different material property information to each region.

Areas such as the sternum, ribs, and collarbone have a thin skin layer and hard tissue, so there is no significant deformation during biopsy. Image segmentation may be additionally performed on the skin area where bone, hard tissue, etc. are located, so that these can be used as a landmark for initial registration.

In order to represent the segmented breast region in a 3D space, volume elements such as tetrahedrons or hexahedrons may be allocated to the breast region. The volume elements can be placed in 3D space to construct a 3D breast model.

Meanwhile, the number of volume elements may affect the update speed of the deformable breast model, so volume elements with different properties may be allocated depending on the required accuracy. For example, when a needle is inserted into the breast, a skin surface area 33 where local deformation occurs or a surrounding area 32 of tissue presumed to be a lesion require higher accuracy, so a greater number of volume elements can be allocated.

As an example, as shown in FIG. 2, a large number of volume elements may be allocated to the area 33 that requires high accuracy. On the other hand, a small number of volume elements may be allocated to the area 34 that requires relatively low accuracy.

As another example, a volume of the volume element allocated based on accuracy may vary. For example, a first volume may be allocated to the volume element of the area 33 where high accuracy is required, and a second volume larger than the first volume may be allocated to the volume element of the area 34 where low accuracy is required. That is, the volume elements are densely arranged in areas where high accuracy is required, and the volume elements are arranged less densely in the remaining areas, thereby increasing computational efficiency.

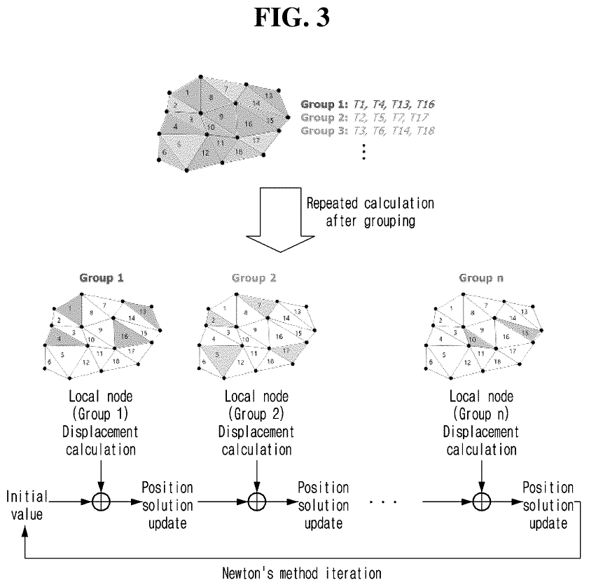

FIG. 3 is another diagram for explaining a method for generating a real-time deformable breast model.

Each volume element constituting the deformable breast model may be given a governing equation based on elastic potential energy. By applying the time integration method to the governing equation, the coordinates of the deformable breast model can be updated at every unit time step. Through updating the coordinates of the deformable breast model, a final deformable breast model can be derived. The governing equation allocated to the volume element may include material parameters of the volume element. For example, the governing equation can be expressed as an equation of motion such as Equation 1 below.

$$M \ddot{x} + ∇ W = {f}_{ext} \qquad [ Equation 1 ]$$

M: Mass of deformable breast model
\dot{x}: Acceleration of deformable breast model
W: Strain energy of deformable breast model
fₑₓₜ: External force applied to deformable breast model

Here, when the deformation characteristics of the deformable breast model follow the Saint Venant-Kirchhoff model, the strain energy W of the deformable breast model in Equation 1 above can be expressed as the following Equation 2.

$$W = V [ {\frac{λ}{2} [ tr ( ε ) ]}^{2} + μ tr ( {ε}^{2} ) ] \qquad [ Equation 2 ]$$

V; Volume of volume element included in deformable breast model
λ, μ: Lame constants

$$λ = Ev / ( 1 + v ) ( 1 - 2 v )$$

$$μ = E / 2 ( 1 + v )$$

E: Young's modulus of deformable breast model
ν: Poisson's ratio of deformable breast model
ε: Green-Lagrange strain tensor

For all volume elements included in the deformable breast model, the strain energy of the entire deformable breast model can be calculated by deriving the strain energy calculated by Equation 2 and adding them all together. In this way, the deformation of the deformable breast model can be calculated similarly to the actual deformation by calculating the strain energy according to the deformation characteristics of the deformable body by considering the Young's modulus (E) and the Poisson's ratio (ν) of the deformable object, which are material parameters.

Since the deformation of the breast is nonlinear, the modeling method used in real-time deformable object simulation can be used to calculate the deformation of the breast model in real time while expressing the nonlinear deformation of the breast. For example, the governing equations can be constructed by adding the corotation method used in real-time object to the governing equations based on the linear finite element method.

In addition, using the non-linear governing equations, calculation speed and efficiency can be improved through repeated updates of the local position solution as shown in FIG. 3.

In addition, material properties can be reflected in deformable breast models by using methods such as xpbd, projective dynamics, and quasi-newton method, which modify the governing equations to add material parameters in real-time deformable object simulations that utilize geometric features such as position-based dynamics (PBD) to speed up calculations.

### Method for Updating Parameters of Deformable Breast Model

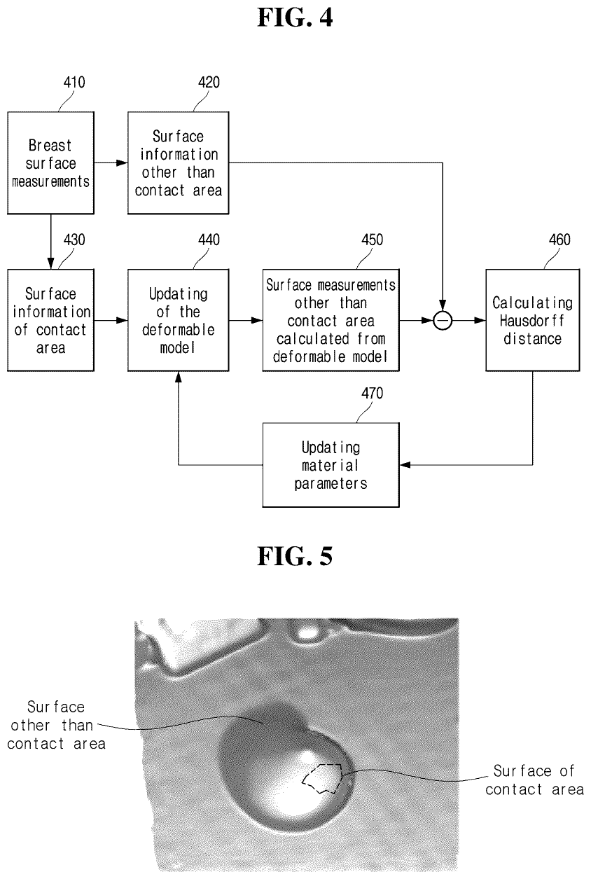

FIGS. 4 and 5 are diagrams for explaining a method of updating parameters of deformable breast model according to another embodiment of the present disclosure.

When a breast biopsy is performed, the surface of the breast can be deformed due to contact with a medical instrument. At this time, real-time 3D surface data of the breast can be used to accurately calculate the deformation of the breast. FIG. 5 shows the surface of a contact area where deformation occurred and a surface other than the contact area being separated.

First, 3D surface data of the breast surface can be measured 410. When deformation occurs due to contact between the medical instrument and the breast surface, the point cloud of the area where the contact occurred can be used as input data for updating and calculating the deformable breast model. Hereinafter, point cloud, surface measurements, and surface information may be used in the same meaning, and the use of information may be used in the same meaning as calculation or operation through information.

3D surface information measurement of the breast surface can be performed using surface information 420 of areas other than the contact area and surface information 430 of the contact area. Initialization and updating 440 of the deformation model may be performed through surface information 430 of the contact area, and surface information 450 other than the contact area calculated from the deformation model may be obtained through the initialized or updated deformation model.

Next, the difference between the surface information 420 of the area other than the contact area where no contact occurred and the surface information 450 of the area other than the contact area calculated in the deformation model is calculated, and feedback can be performed to update 440 the deformable model. This feedback can be performed by updating (470) the material parameters of the deformable breast model in a direction that reduces the difference between the surface information 420 of the area other than the contact area where no contact occurs and the surface information 450 of the area other than the contact area calculated in the deformation model. In the previous description, differences in surface information may mean differences in coordinate values of each surface.

The Hausdorff distance calculation method used in real-time interaction simulation may be utilized 460 to calculate the difference between surface information 420 other than the contact area where no contact occurred and surface information 450 other than the contact area calculated from the deformation model. As optimization method for material parameters update 470, one of a genetic algorithm, an EM algorithm, or techniques used for optimization in real-time simulation may be used.

### Method for Measuring Real-Time Breast Shape Using Depth Sensor

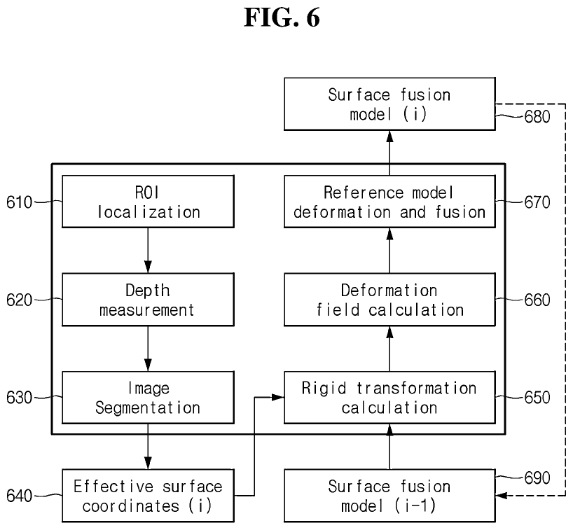

FIG. 6 is a diagram for explaining a method of measuring breast shape in real time using a depth sensor according to another embodiment of the present disclosure.

First, localization of a region of interest (ROI) for performing real-time shape measurement may be performed (610). ROI localization can be performed using machine learning or image processing algorithms. Through ROI localization, an object of interest can be localized within a confined ROI in real time, and the amount of computation is reduced, which can improve the operation speed of the entire algorithm.

Next, the 3D surface data of the object existing in the effective image area may be measured in real time by the depth sensor (620).

Next, using an image processing algorithm, machine learning algorithm, or statistical outlier removal technique using the brightness value for each RGB channel and the brightness ratio between channels, information 640 on the object of interest among the 3D surface coordinates of the object calculated within the effective image area may be extracted and calculated in real time (630). Here, the object of interest may be the patient's breast.

Next, a 3D rigid transformation can be calculated between a 3D surface data of the measured breast and a reference model. Here, the reference model may mean the 3D surface data 690 of the surface fusion model in the previous time unit. The calculated 3D rigid transformation can be used to translate and rotate the surface data measured in the sensor coordinate system to the model coordinate system (650).

Next, a deformation field may be calculated using a node generated by sampling data from the transformed surface data and the reference model (660). At this time, the transformation field may mean a 3D rigid transformation for each node.

Next, a surface fusion model 680 may be generated by fusing the reference model to which the deformation field is applied and the surface data (670). Afterwards, steps 610 to 690 are repeated per unit of time, so that the surface fusion model can be updated in real time.

Depth sensor measurements or surface information measured at various angles may be fused to generate a surface fusion model. That is, surface information measured at various angles can be stitched to generate a surface fusion model.

When a surgical procedure is in progress, shape prediction may be possible even in blind spots where the field of view is obscured by the operator's hands, medical instruments, etc. The 3D position and rotation components of the blind spot nodes can be interpolated as a weighted sum of nearby nodes sampled from the 3D surface model. Since confidence and weight for each node can be cumulatively added for each time unit, the operator's hands and medical instruments that enter the operator's field of view may be recognized as objects of non-interest and may not affect breast surface information.

To implement the above-described blind spot deformation presuming method, the depth sensor can be swept using the operator's hand or a robot in the initial stage of the procedure. Based on this, all surface areas without blind spots can be measured and stored in the database. Information about the surface area measured here can be used to generate a surface fusion model at a later stage.

By adjusting parameters such as the number of sampling nodes and the distance between nodes, more precise shape prediction and improved update speed can be achieved. Additionally, the degree of deformation between the previous unit time and the current unit time can be calculated and visualized in real time.

Additionally, the degree of deformation between the previous unit time and the current unit time can be calculated and visualized in real time for each area of the surface fusion model. By adjusting the deformation field calculation parameters such as the number of sampling nodes and the distance between nodes based on this information, the spatial precision or resolution of shape prediction can be selectively increased only for local areas where large deformation occurs. On the other hand, for other areas, the accuracy and speed of shape measurement can be improved by lowering the spatial precision or resolution of the shape prediction space.

A robot arm equipped with a depth sensor may move based on the real-time deformation measurement data described above. At this time, the depth sensor or robot arm equipped with the depth sensor can automatically search for and move to a position that can best measure breast deformation. Based on the movement of the depth sensor, obstruction of the field of view caused by the operator's hands or medical instruments can be alleviated.

### Real-Time Image-to-Patient Registration Method

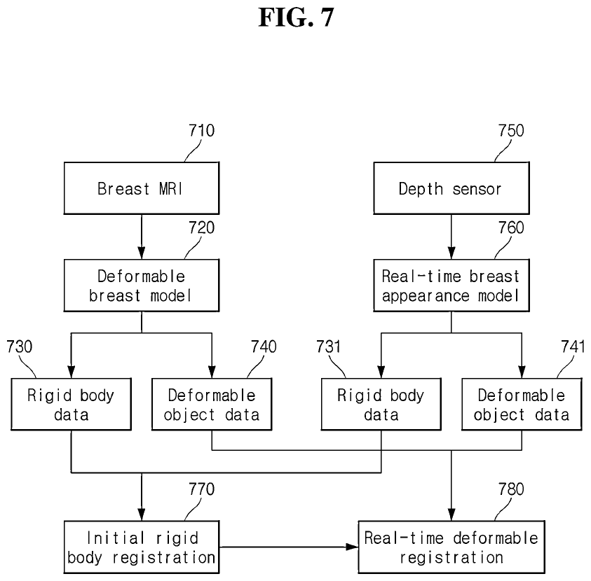

FIG. 7 is a diagram for explaining a real-time image-to-patient registration method according to another embodiment of the present disclosure.

According to another embodiment of the present disclosure, real-time image-to-patient registration can be performed using a deformable breast model and real-time 3D breast surface measurement data. The real-time image-to-patient registration method may be performed by initial rigid-body registration 770 and real-time deformable registration 780.

The breast MRI 710 may generate a deformable breast model 720 using at least one of the above-described embodiments, and the depth sensor 750 may generate a 3D breast surface model 760 in real time.

Initial rigid-body registration 770 may be performed on areas where the skin layer is thin and the bone is located, so the deformation is relatively small such as the sternum, ribs, and collarbone. Data for the initial rigid-body registration 770 may be obtained through data 730 obtained from an MRI data before the medical procedure and data 731 corresponding to patient skin data measured using the depth sensor 750 during the medical procedure. Rigid-body registration can be performed using the two data 730 and 731 described above.

Meanwhile, real-time deformable registration 780 can be performed through deformable breast model 740 generated using MRI data before the medical procedure and breast surface fusion model 741 generated using the surface information obtained from the depth sensor in real time during the medical procedure.

Real-time deformable registration 780 may be performed repeatedly during the medical procedure. Meanwhile, rigid-body registration may not be performed periodically during the medical procedure but may be performed intermittently under some circumstances. For example, rigid-body registration may be performed when an unexpected patient movement occurs or when there is a significant change in the surgical environment.

### Indirect MRI-Guided Breast Biopsy Apparatus and System

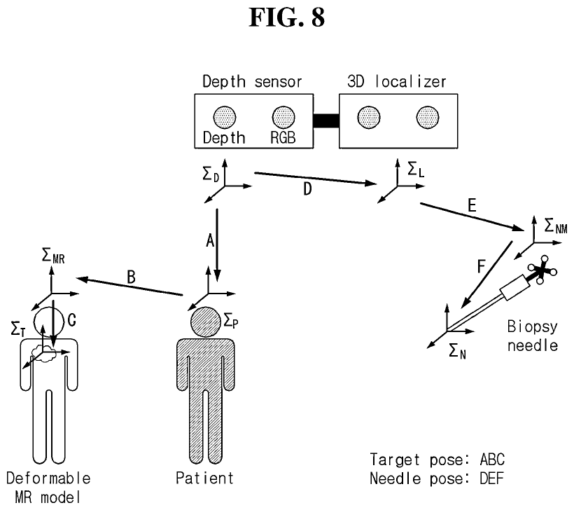

FIGS. 8 to 11 are diagrams for explaining an apparatus and system for an indirect MRI-guided breast biopsy according to some embodiments of the present disclosure.

Referring to FIGS. 8 to 11, the apparatus and system for an indirect MRI-guided breast biopsy capable of performing the above-described method for the indirect MRI-guided breast biopsy will be described. In FIGS. 8 to 11, character strings written in capital letters may mean coordinate system information possessed by each element. The coordinate system information can also be implemented in the form of a transformation matrix.

### Apparatus and System for Indirect MRI-Guided Breast Biopsy Using Virtual Reality Apparatus

FIG. 8 illustrates an apparatus and system for an indirect MRI-guided breast biopsy using a virtual reality apparatus. The embodiment shown in FIG. 8 may be implemented using at least one of a method for generating a deformable breast model, a method for updating deformable breast model parameters, a method for measuring breast shape using a depth sensor, and a real-time deformable registration method.

The position and orientation of medical instruments tracked by a 3D position tracking apparatus and deformable breast model and MRI targets tracked by depth sensor can be displayed in real time through an external monitor.

As an example, a 3D position tracking apparatus may be composed of at least one of an optical position tracking apparatus and a position tracking apparatus using an electromagnetic field. As another example, when the 3D position tracking apparatus is a position tracking apparatus consisting of a single or stereo depth sensor and the tracking apparatus uses single or stereo RGB camera built into the depth sensor, the depth sensor may simultaneously perform the role of the 3D position tracking apparatus.

The user or operator can perform a breast biopsy while checking the real-time position of the biopsy needle and the position of the MRI target displayed on the external display.

The following Equation 3 is an equation for explaining the symbols shown in FIG. 8.

### [Equation 3]

Σ_{D}: RGB-D sensor coordinate system
Σ_{L}: Localizer coordinate system
Σ_{P}: Patient coordinate system
Σ_{MR}: MRI coordinate system
Σ_{T}: Target coordinate system
Σ_{NM}: Needle maker coordinate system
Σ_{N}: Needle coordinate system

### Apparatus and System for Indirect MRI-Guided Breast Biopsy Using Augmented Reality Apparatus

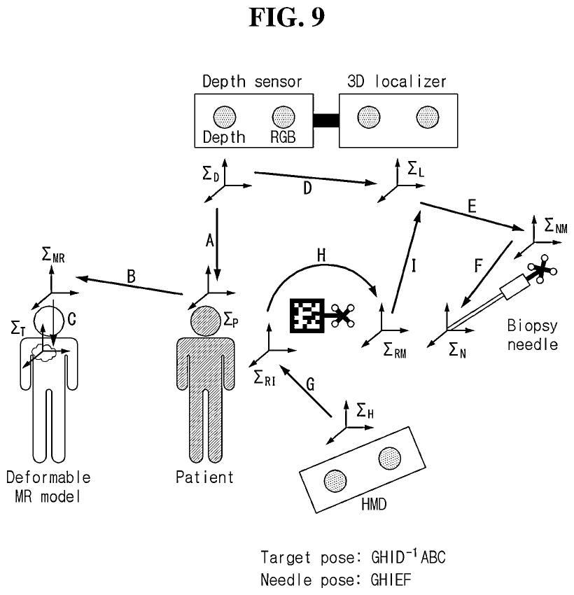

FIG. 9 shows an apparatus for an indirect MRI-guided breast biopsy using an augmented reality apparatus. The apparatus for an indirect MRI-guided breast biopsy according to this embodiment can be configured by adding a reference apparatus (ERM) including a 3D position tracking marker and an HMD tracking marker to the apparatus for an indirect MRI-guided breast biopsy shown in FIG. 8.

Here, the 3D position tracking marker may be a marker that can track the position and orientation of an object using a 3D position tracking apparatus. The 3D position tracking marker may be one of an infrared retroreflective marker, an electromagnetic field marker, a QR code, and an AR marker. Meanwhile, the HMD tracking marker is a marker that can be identified by the HMD and may be one of an infrared retroreflection marker, a QR code, or an AR marker.

Meanwhile, when a QR code is used as a 3D position tracking marker and an HMD tracking marker, the QR code may additionally include information on the patient on whom the surgical procedure is performed.

The 3D position tracking marker and the HMD tracking marker can be physically fixed to form one apparatus. Additionally, the 3D rigid transformation between the 3D position tracking marker and the HMD tracking marker coordinate systems can be estimated through a one-time calibration procedure.

The virtual reality information described in FIG. 8 can be provided to a user or operator wearing the HMD using a reference apparatus that can be identified by the HMD and the 3D position tracking apparatus, and the user or operator can perform a breast biopsy using only the images and information displayed on the HMD. At this time, the breast biopsy may be performed remotely in a space that is not the same as the patient. Specifically, the user or operator can perform a breast biopsy while checking the real-time position of the biopsy needle and the position of the MRI target displayed on the HMD.

The following Equation 4 is an equation for explaining the symbols shown in FIG. 9.

[Equation 4]
Σ_{D}: RGB-D sensor coordinate system
Σ_{L} Localizer coordinate system
Σ_{P}: Patient coordinate system
Σ_{MR}: MRI coordinate system
Σ_{T}: Target coordinate system
Σ_{NM}: Needle maker coordinate system
Σ_{N}: Needle coordinate system
Σ_{RI}: Reference image coordinate system
Σ_{RM}: Reference marker coordinate system
Σ_{H}: HMD coordinate system

### Apparatus for Indirect MRI-Guided Breast Biopsy Using Robotic Arm

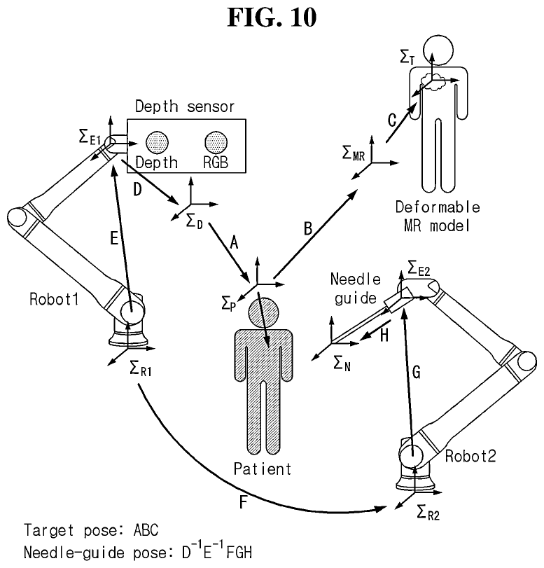

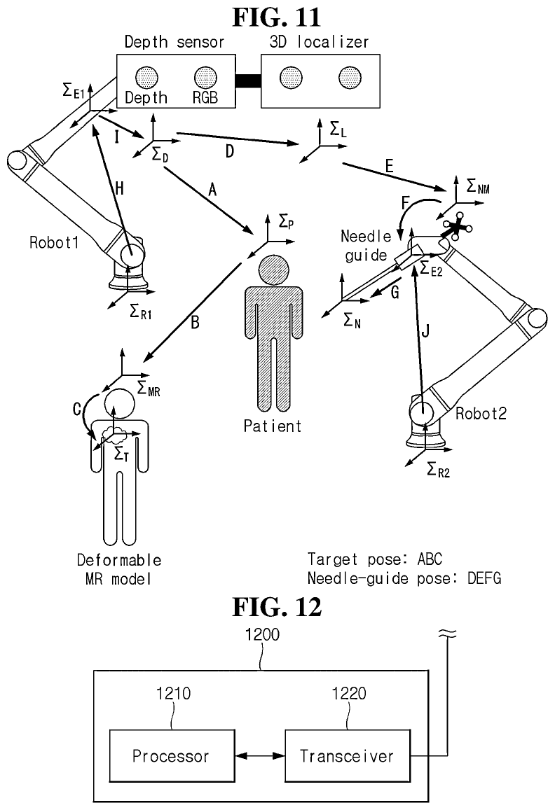

FIGS. 10 and 11 illustrate an apparatus for an indirect MRI-guided breast biopsy using a robot or robotic arm. The embodiment shown in FIGS. 10 and 11 may be implemented using at least one of a method for measuring breast shape in real time using the depth sensor described above, a method for updating deformable breast model parameters, and a real-time deformable registration method.

According to the embodiment of FIG. 10, the forward kinematics for each robot arm can be calculated to calculate the position and orientation of the end effector of the robot arm with respective to the robot base coordinate system. A depth sensor (Σ_{D}) may be attached to the end effector (Σ_{E1}) of the first robot arm. The real-time registration (B) between the patient and the deformable breast model can be performed using the shape sensing data (A) of the breast surface measured by the depth sensor. Based on real-time deformable registration, the apparatus for an indirect MRI-guided breast biopsy can estimates the real-time position and orientation (ABC) of the MRI target (Σ_{T}) with respect to the depth sensor coordinate system in real time.

The rigid transformation (D) between the coordinate system (Σ_{E1}) of the first robot's end effector and the coordinate system (Σ_{D}) of the depth sensor can be estimated by Hand-eye calibration.

By moving the first robot arm at various angles, a surface fusion model without blind spots can be generated. The first robot arm can move in real time to measure breast shape, updating a deformable breast model in real time.

The rigid transformation (F) between the two coordinate systems of the robot bases can be calculated through one-time calibration.

A needle guide apparatus (Σ_{N}) through which a biopsy needle can be inserted may be attached to the end effector of the second robot arm. The position and orientation of the needle guide apparatus based on the coordinate system of the depth sensor can be calculated through D⁻¹E⁻¹FGH. Here, H can be calculated through calibration of the guide apparatus, and G can be calculated by solving the forward kinematics of the second robot arm.

The motion of the second robotic arm may be adjusted to move the needle guide apparatus (Σ_{N}) to the real-time position and orientation (ABC) of the MRI target described with respected to the depth sensor coordinate system. At this time, the operation of the second robot arm can be controlled by reflecting the procedure plan for each patient.

At this time, the user can collect a tissue sample from the MRI target using a biopsy needle according to the guidance of the guide apparatus mounted on the second robot arm.

The following Equation 5 is an equation for explaining the symbols shown in FIG. 10.

### [Equation 5]

Σ_{R1}: Robot1 base coordinate system
Σ_{E1}: Robot1 end-effector coordinate system
Σ_{R2}: Robot2 base coordinate system
Σ_{E2}: Robot2 end-effector coordinate system
Σ_{ZD}: GB-D sensor coordinate system
Σ_{P}: Patient coordinate system
Σ_{MR}: MRI coordinate system
Σ_{T}: Target coordinate system
Σ_{N}: Needle coordinate system

According to the embodiment of FIG. 11, the forward kinematics of each robot arm can be solved to calculate the position and orientation (H, J) of each end effector of both robot arms with respective to the robot base coordinate systems.

At least one of a depth sensor (Σ_{D}) and a 3D position tracking apparatus (Σ_{L}) may be physically mounted on the end effector (Σ_{E1}) of the first robot arm. The real-time position of the MRI target with respective to the depth sensor coordinate system can be calculated by measuring the shape (A) of the breast surface using the depth sensor and performing image-to-patient registration (B) using the deformable model in real time.

The position and orientation (I) between the coordinate system (Σ_{E1}) of the first robot's end effector and the coordinate system (Σ_{D}) of the depth sensor can be estimated by Hand-eye calibration.

The first robot arm moves at various angles, so that a surface fusion model without blind spots can be generated. The first robot arm can move in real time to measure breast shape, updating a deformable breast model in real time.

A needle guide apparatus (Σ_{N}) for inserting a biopsy needle and a 3D position tracking marker (Σ_{NM}) may be attached to the end effector (Σ_{E2}) of the second robot arm. The position and orientation of the needle guide apparatus with respective to the coordinate system of the depth sensor can be calculated with DEFG using information from the 3D position tracking apparatus and can be adjusted by solving the inverse kinematics (J⁻¹) of the second robotic arm.

The motion of the second robot arm (J) may be adjusted so that the needle guide apparatus (Σ_{N}) moves to the real-time position and orientation (ABC) of the MRI target with respective to the depth sensor coordinate system. At this time, the operation of the second robot arm can be controlled by reflecting the procedure plan for each patient.

At this time, the user can collect a tissue sample from the MRI target using a biopsy needle according to the guidance of the guide apparatus mounted on the second robot arm.

The following Equation 6 is an equation for explaining the symbols shown in FIG. 11.

### [Equation 6]

Σ_{R1}: Robot1 base coordinate system
Σ_{E1}: Robot1 end-effector coordinate system
Σ_{R2}: Robot2 base coordinate system
Σ_{E2}: Robot2 end-effector coordinate system
Σ_{D}: RGB-D sensor coordinate system
Σ_{L}: Localizer coordinate system
Σ_{P}: Patient coordinate system
Σ_{MR}: MRI coordinate system
Σ_{T}: Target coordinate system
Σ_{NM}: Needle maker coordinate system
Σ_{N}: Needle coordinate system

FIG. 12 is a diagram illustrating a configuration of an apparatus according to an embodiment of the present disclosure.

FIG. 12 is a diagram illustrating an apparatus configuration, according to an embodiment of the present disclosure. Referring to FIG. 12, the apparatus may be configured using at least one of the above-described apparatus for an indirect MRI-guided breast biopsy, a depth sensor, a position tracking apparatus, and one or more robot arms. The apparatus may be an apparatus that communicates with and is interoperable with other apparatus, and is not limited to the above-described embodiments. As an example, the apparatus 1200 may include at least one of a processor 1210 and a transceiver 1220 for the above-described operation. That is, the apparatus may include the configuration necessary to communicate with other apparatus.

As an example, the apparatus 1200 may include a processor 1210 and a transceiver 1220 for the above-described operations. That is, the apparatus may include the configuration necessary to communicate with other apparatus. Additionally, as an example, the apparatus may include other components, such as memory, in addition to the components described above. In other words, the apparatus is only a configuration that includes the above-described apparatus to communicate with other apparatus, but is limited thereto, and may be an apparatus that operates based on the above-described apparatus.

Although the above-described exemplary methods of the present disclosure are expressed as a series of operations for clarity of explanation, this is not intended to limit the order in which the steps are performed, and if necessary, each step may be performed simultaneously or in a different order. In order to implement the method according to the present disclosure, other steps may be included in addition to the exemplified steps, some steps may be excluded, and the remaining steps may be included, or some steps may be excluded, and additional other steps may be included.

The various embodiments of the present disclosure do not list all possible combinations, but are intended to explain representative aspects of the present disclosure, and matters described in the various embodiments may be applied independently or in combination of two or more.

Additionally, various embodiments of the present disclosure may be implemented by hardware, firmware, software, or a combination thereof. For hardware implementation, it may be implemented by one or more Application Specific Integrated Circuits (ASICs), Digital Signal Processors (DSPs), Digital Signal Processing Devices (DSPDs), Programmable Logic Devices (PLDs), Field Programmable Gate Arrays (FPGAs), general processors, controllers, microcontrollers, microprocessors, etc.

The scope of the present disclosure includes software or machine-executable instructions (e.g. operating system, applications, firmware, programs, etc.) that cause operations according to the methods of various embodiments to be executed on a device or computer, and non-transitory computer-readable medium in which such software or instructions are stored and executable on a device or computer.

What is claimed is:

1. A method for a breast biopsy based on indirect magnetic resonance imaging (MRI) guidance comprising:
obtaining a breast MRI data of a patient;
generating a deformable breast model using the breast MRI data;
measuring real-time breast shape of the patient using a depth sensor;
performing a real-time deformable registration using the deformable breast model and the real-time shape data;
estimating movements of the MRI targets inside of the breast;
performing a breast biopsy for the MRI targets.

2. The method of claim 1, wherein the generation of the deformable breast model includes:
segmenting breast tissue of the patient from the breast MRI data; and
representing the breast of the patient as a three-dimensional (3D) model by allocating a plurality of volume elements to the segmented breast tissue.

3. The method of claim 2, wherein different numbers of volume elements are allocated to each area of the breast tissue based on accuracy required for an examination.

4. The method of claim 2, wherein each of the plurality of volume elements is given a governing equation based on elastic potential energy, and
the governing equation includes material parameters of the breast tissue.

5. The method of claim 1, further comprising:
updating material parameters of the deformable breast model in real time based on interaction between a medical instrument and a breast of the patient,
wherein the update is performed based on a contact area between the medical instrument and the breast.

6. The method of claim 5, wherein the material parameters of the deformable breast model are updated based on a difference between first surface information measured in real time in an area other than the contact area and second surface information calculated from an area other than a contact area on the deformable breast model.

7. The method of claim 6, wherein the difference between the first surface information and the second surface information is calculated based on Hausdorff distance.

8. The method of claim 1, wherein the real-time deformable breast model is updated based on information collected about a breast of the patient from various angles by the depth sensor.

9. The method of claim 8, wherein the information collected about the breast of the patient from various angles includes information about blind spots where visibility is obscured.

10. The method of claim 8, wherein the information collected about the breast of the patient from various angles is obtained by changing a position and orientation of the depth sensor in real time.

11. The method of claim 1, further comprising:
performing deformable registration between the deformable breast model generated from the MRI data and the real-time breast shape data measured by the depth sensor.

12. The method of claim 11, wherein the registration between the two data is performed separately into rigid-body registration and real-time deformation registration.

13. The method of claim 1, wherein the measuring real-time breast shape is performed by the depth sensor acquiring breast surface information of the patient and generating and updating a deformable surface fusion model in real time.

14. The method of claim 13, wherein the deformable surface fusion model is obtained by calculating a degree of deformation between a previous unit time and a current unit time for each area.

15. The method of claim 14, wherein the deformable surface fusion model is obtained by giving higher spatial resolution to areas with a large degree of deformation than to areas with a relatively small degree of deformation.

16. An apparatus for an indirect MRI-guided breast biopsy comprising:
a transceiver configured to transmit and receive a signal;
a processor configured to control the transceiver,
wherein the processor is configured to:
obtain a breast MRI data of a patient;
generate a deformable breast model using the breast MRI data;
measure real-time breast shape of the patient using a depth sensor;
perform a real-time deformable registration using the deformable breast model and the real-time shape data;
estimate movements of the MRI targets inside of the breast;
perform a breast biopsy for the MRI targets.

17. The apparatus of claim 16, wherein the deformable breast model is generated by segmenting breast tissue of the patient from the breast MRI data, and
representing the breast of the patient as a 3D model by allocating a plurality of volume elements to the segmented breast tissue.

18. The apparatus of claim 17, wherein different numbers of volume elements are allocated to each area of the breast tissue based on accuracy required for an examination.

19. The apparatus of claim 16, wherein the processor updates material parameters of the deformable breast model in real time based on interaction between a medical instrument and a breast of the patient,
wherein the update is performed based on a contact area between the medical instrument and the breast.

20. The apparatus of claim 16, wherein the real-time deformable breast model is updated based on information collected about a breast of the patient from various angles by the depth sensor.
